(19) United States
(12) Patent Application Publication
(10) Pub. No.: US 2016/0122820 A1
(43) Pub. Date: May 5, 2016

(54) Screening Method for Identifying Active Agents

(71) Applicant: Avon Products, Inc., Suffern, NY (US)

(72) Inventors: Yong Zhuang, Livingston, NJ (US); Russell J. Wyborski, Pine Island, NY (US); Permanan Raaj Khusial, Highland Mills, NY (US); Daniel Thorn Leeson, Chatam, NJ (US)

(21) Appl. No.: 14/800,847

(22) Filed: Jul. 16, 2015

Related U.S. Application Data
(60) Provisional application No. 62/072,012, filed on Oct. 29, 2014.

(51) Int. Cl. C12Q 1/68; A61Q 19/06; A61Q 19/08; A61Q 19/00; A61K 8/36; A61K 8/34

(52) U.S. Cl. CPC C12Q 1/6883; A61K 8/361; A61K 8/342; A61Q 19/08; A61Q 19/00; A61Q 19/007; A61Q 19/06; C12Q 2600/136; C12Q 2600/158

(57) ABSTRACT

The present invention relates generally to screening methods, and in particular, screening methods for identifying active agents having therapeutic or cosmetic benefits to the human integumentary system. The invention also relates to compositions for topical application to the skin comprising active agents identified by the screening methods of the invention, and to methods for improving the health and/or appearance of skin by topically administering to the skin compositions comprising active agents identified by the screening methods of the invention.

### CROSS-REFERENCE TO RELATED APPLICATIONS

This patent application claims priority to U.S. Patent Application Ser. No. 62/072,012, filed on Oct. 29, 2014. The entirety of the aforementioned application is incorporated herein in its entirety by reference.

### FIELD OF INVENTION

The present invention relates generally to screening methods, and in particular, screening methods for identifying active agents having therapeutic or cosmetic benefits to the human integumentary system. The invention also relates to compositions for topical application to the skin comprising active agents identified by the screening methods of the invention, and to methods for improving the health and/or appearance of skin by topically administering to the skin compositions comprising active agents identified by the screening methods of the invention.

### BACKGROUND OF THE INVENTION

Skin aging is a continuous, multifactorial process that is caused by intrinsic biological processes such as genetics and metabolic processes, as well as by environmental factors such as oxidative damage from overexposure to ultraviolet (UV) sunlight. Histological studies of the skin show that as aging occurs, the skin undergoes structural, functional, and metabolic changes that parallel the aging and degenerative changes in other body organs. As a result, the surface of aged or aging skin may exhibit fine lines and wrinkles, sagging, dullness, discoloration, uneven tone, rough texture, loss of thickness, and the like.

Consumers are interested in mitigating, delaying, or reversing the dermatological signs of chronologically- or hormonally-aged skin, as well as skin aging due to environmental stress. Although numerous products are currently sold to combat the appearance of aging skin, the cosmetics industry is actively pursuing new active agents that may be used to reduce signs of aging or aged skin (e.g., wrinkles and fine lines).

It is therefore an object of the invention to provide new methods of screening for active agents that can be used to treat the manifestations of skin aging and/or improve skin health. It is a further object of the invention to improve the overall aesthetic appearance of skin, including treating, ameliorating, reversing, and/or preventing signs of skin aging or damage, by topically treating skin with active agents identified by the screening methods of the invention.

The foregoing discussion is presented solely to provide a better understanding of the nature of the problems confronting the art and should not be construed in any way as an admission as to prior art nor should the citation of any reference herein be construed as an admission that such reference constitutes “prior art” to the instant application.

### SUMMARY OF THE INVENTION

The present invention is based on the discovery of a unique set of genes associated with skin health and/or appearance, and the recognition that this set of genes (or any subset thereof) is useful for identifying new skin active agents. The genes were identified through extensive study of human skin treated with three treatment modalities, each of which is documented to be effective: laser treatment, retinol, and retinoic acid. To determine which genes are regulated in response to all three of these treatments, gene expression was measured in skin following application of each of the treatments to the skin. Gene array analyses were conducted on mRNA isolated from the skin following exposure to laser treatment, retinol, or retinoic acid treatment, and gene expression was measured. Gene expression was also measured from mRNA isolated from the skin prior to any treatment, to establish baseline gene expression. Gene expression was considered modulated (whether upregulated or downregulated) if there was a change in expression relative to baseline of at least 1.3-fold following treatment. A unique subset of 59 genes (see Table 1) was modulated following all three of the treatments. This unique group of genes provides a gene signature for identifying new actives. Specifically, whether a candidate substance may be useful as a skin active may be determined by contacting a skin cell (e.g., fibroblast, keratinocyte, and/or melanocyte) with the candidate substance and determining whether any of the genes in Table 1 (e.g., two or more, three or more, four or more, etc.) are differentially expressed (i.e., upregulated or downregulated).

Accordingly, in one aspect of the invention, a method is provided for screening for candidate substances to identify actives useful for improving the health and/or appearance of skin. The method generally comprises contacting a human skin cell (e.g., fibroblast, melanocytes, keratinocyte) with a candidate substance and measuring expression levels of at least two genes (e.g., at least 3 genes, at least 4 genes, at least 5 genes, at least 10 genes, etc.) selected from those listed in Table 1 relative to control (e.g., otherwise identical cells treated with a vehicle in the absence of the candidate substance), in the absence of the candidate substance. In other embodiments, the candidate substance may be topically applied to skin (in vivo or ex vivo) to assess the modulation of those genes. It may then be determined if the expression of at least two of the measured genes is modulated (e.g., upregulated or downregulated) following contact with the candidate substance, relative to baseline (control). Expression of the genes in Table 1 may be assessed, although expression of any homolog, fragment or marker of the genes listed in Table 1 may be measured as well. Modulation (e.g., about a 1.5-fold, 1.3-fold, 2-fold, 3-fold, 4-fold, 5-fold, 10-fold, 12-fold, 15-fold, etc. change in expression relative to baseline) of two or more genes selected from Table 1 indicates that the candidate substance is an active suitable for improving the health and/or appearance of skin (e.g., treating/preventing fine lines or wrinkles, improving skin thickness or plumpness, improving skin tone, improving hyper- or hypo-pigmentation, etc.). The method may also comprise extracting nucleic acids (e.g., mRNA, DNA, etc.) from skin cells that have not been and skin cells that have been exposed to a candidate substance. Typically, complementary nucleic acids (e.g., cDNA, cRNA, etc.) are synthesized from the extracted nucleic acids in enzyme catalyzed reactions by methods known to those skilled in the art. Gene expression levels may then be quantified using methods routine to those skilled in the art (e.g., quantitative PCR, Northern blot assays, RNA protection assays, etc.). In some aspects, the method involves measuring gene expression levels using a gene array. When a gene array is used to assess gene expression, the complementary nucleic acids may be contacted with a solid phase gene panel (e.g., comprising glass, silicon, nylon, etc.) having immobilized thereon a plurality of oligonucleotides (e.g., primers or probes) that hybridize to nucleic acid sequences corresponding to two or more (e.g., three or more, four or more, five or more, etc.) genes selected from Table 1. The hybridization may then be detected and quantified by methods known in the art (e.g., using fluorophore-labelled cDNA or cRNA) to quantify gene expression levels.

In some implementations of the invention, expression of at least one (e.g., at least two, at least three, at least four, at least five, etc.) of the genes modulated in skin cells following exposure of the skin cell to a candidate substance is selected from: sterile alpha motif domain containing 9; UDP glucuronosyltransferase 2 family, polypeptide A1, complex locus; cytohesin 1 interacting protein; interferon-induced protein 44; interferon-induced protein 44-like; ecotropic viral integration site 2B; G protein-coupled receptor 174; secreted phosphoprotein 1; GLI pathogenesis-related 1; protein tyrosine phosphatase, receptor type, C; and placenta-specific 8. In another aspect, at least one of the genes modulated in skin cells following exposure of the skin cell to a candidate substance is selected from collagen, type XIV, alpha 1; collagen, type V, alpha 2; collagen, type III, alpha 1; collagen, type VI, alpha 6; and fibroblast activation protein, alpha. In another aspect, at least one of the genes modulated in skin cells following exposure of the skin cell to a candidate substance is phosphoprotein 1.

In yet another aspect of the invention, a gene array is provided for screening for active agents. The gene array typically comprises a solid phase (e.g., comprising glass, silicon, nylon, etc.) having immobilized thereon a plurality of oligonucleotides (e.g., primers, probes, etc.). At least some of the oligonucleotides hybridize to nucleic acids sequences corresponding to or complementary to two or more genes listed Table 1. In some aspects, a gene array (e.g., gene chip, biochip) is provided wherein at least 50%, (e.g., at least 60%, at least 70%, at least 80%, at least 85%, at least 90%, at least 95%, or at least 99%) of the nucleic acids on the gene panel comprise oligonucleotides (e.g., primers, probes that may be less than about 60, less than about 50, less than about 40, less than about 30, less than about 20, or less than about 10 nucleotides in length) that hybridize to nucleic acids corresponding to or complementary to those listed in Table 1. In some implementations, the gene array will comprise nucleic acids that correspond to or are complementary to at least two, or at least three, or at least four, or at least five, or at least six, or at least seven, or at least eight, or at least nine, or at least ten genes from Table 1. In other implementations, the gene array will comprise at least fifteen, or at least twenty, or at least twenty-five, or at least thirty, or at least thirty-five, or at least forty, or at least forty-five, or at least fifty genes from Table 1.

In another aspect of the invention, topical compositions (e.g., in a dermatologically or physiologically acceptable vehicle) are provided that comprise one or more active agents identified by the screening methods of the invention, or identified using the gene arrays of the invention. In yet another aspect of the invention, methods for improving the health and/or appearance of skin (e.g., treating/preventing fine lines or wrinkles, improving skin thickness or plumpness, improving skin tone, improving hyper- or hypo-pigmentation, treating signs of skin aging, etc.) are provided, comprising topically applying to an area of the skin in need thereof a composition comprising one or more actives identified according to the screening methods of the invention, or identified using the gene arrays of the invention.

These and other aspects of the present invention will be better understood by reference to the following detailed description and appended claims.

### DETAILED DESCRIPTION

All terms used herein are intended to have their ordinary meaning unless otherwise provided. All ingredient amounts provided herein are by weight percent of the total composition unless otherwise indicated. As used herein, the term “consisting essentially of” is intended to limit the invention to the specified materials or steps and those that do not materially affect the basic and novel characteristics of the claimed invention, as understood from a reading of this specification.

As used herein, the term “expression levels” refers to an amount of a gene and/or protein that is expressed in a cell (e.g., number of mRNA copies made). As used herein, a “gene” includes a polynucleotide containing at least one open reading frame that is capable of encoding a particular polypeptide. As used herein, the term “polynucleotide” includes “oligonucleotide” and includes polymeric forms of nucleotides (nucleic acids) of any length, either deoxyribonucleotides or ribonucleotides, or analogs thereof, including, without limitation, mRNA, DNA, cDNA, cRNA, microRNA, primers, probes, and the like.

The present invention is based on the discovery that new skin actives can be identified based on their ability to modulate the expression of a novel set of genes. This novel set of genes is useful for identifying agents that can improve the health and/or appearance of skin (e.g., treating/preventing one or more dermatological signs of aging, such as treating/preventing fine lines or wrinkles, improving skin thickness or plumpness, improving skin tone, improving hyper- or hypo-pigmentation, treating signs of skin aging, etc.).

In one embodiment, a method is provided for screening for candidate substances to identify active agents useful for improving the health and/or appearance of skin. The method generally comprises contacting a human skin cell with a candidate substance, and measuring the expression levels of two or more (e.g., three or more, four or more, five or more, etc.) genes selected from the group consisting of the genes listed in Table 1 or any subset thereof, relative to expression levels of the same genes in a control sample (baseline), by which is meant otherwise identical cells treated with a vehicle in the absence of the candidate substance. In other embodiments, the candidate substance may be topically applied to skin (in vivo or ex vivo) to assess the modulation of those genes. Modulation of genes (or any homolog, fragment or marker of the genes) in Table 1 indicates that the candidate substance is an active that is suitable for improving the health and/or appearance of skin.

Typically, the screening is carried out in vitro. Any suitable human skin cells may be used to assay the candidate substance, such as, for example, fibroblasts, melanocytes, and/or keratinocytes. The cells are typically cultured in the presence of the candidate substance. The candidate substance may be, for example, an antibody, a nucleic acid (e.g., siRNA), a small molecule, a botanical extract, etc.

In embodiments where the candidate is topically applied to skin, any portion of human skin tissue may be contacted with a candidate substance to assess gene expression levels in that tissue, such as skin from the face, neck, arms, legs, buttocks, chest, etc. The skin cells may be obtained from skin that is aged, and has, for example, wrinkles, fine lines, hyperpigmentation, thickening, uneven skin tone, etc., or from skin that is not aged and does not possess the foregoing characteristics. Gene expression may be measured from any skin cell type, such as, for example, fibroblasts melanocytes, or keratinocytes.

The candidate substance may be applied to the skin topically, for example in the form of a cream, lotion, ointment, patch, etc. In some embodiments, the candidate substance will be applied to the skin at least once daily (or twice daily) for at least 1 day, or at least 2 days, or at least 7 days, or at least 14 days, or at least four weeks, or at least 8 weeks. The candidate substance will be in contact with the skin for a sufficient length of time to provide a measurable change in gene expression levels, which will typically be at least half an hour, at least one hour, and more typically from about 12 hours to about 72 hours. However, in some embodiments, the candidate substance may be in contact with the skin for about one or more weeks, or for about one or more months. In some embodiments, other forms of skin treatment may be assessed by the screening methods of the invention, such as laser treatments, thermolysis treatments, electromagnetic radiation, or other treatments involving use of current, such as radio frequency current.

Following treatment with a candidate substance, a biological sample, such as a sample of the treated skin, may be obtained. A biological sample may include any sample of biological tissue or fluid that comprises nucleic acids. Skin cells may be obtained from at least one portion of the treated skin, for example, by biopsy (e.g., punch biopsy), by fine-needle aspiration, by cell scraping, etc., so that nucleic acids may be extracted from the biological sample.

Nucleic acids (e.g., mRNA, DNA) may be extracted from biological samples by any conventional methods, so that gene expression levels may be measured. Typically, prior to gene expression quantification procedures, complementary nucleic acids (e.g., cDNA or cRNA) are synthesized from the extracted nucleic acids in enzyme catalyzed reactions by methods known to those skilled in the art.

Gene expression levels may be measured by any suitable technique for detection and quantitation of polynucleotides (e.g., mRNA, DNA, microRNA), such as, for example, quantitative polymerase chain reaction (QPCR), real-time QPCR, reverse transcription PCR (RT-PCR), as are well-known in the art. As described in detail in U.S. Pat. Nos. 7,101,663 and 7,662,561, the disclosures of which are hereby incorporated by reference, a quantitative reverse transcriptase polymerase chain reaction (QRT-PCR) for detecting mRNA may include the steps of: (a) incubating an RNA sample from the cellular lysate with a reverse transcriptase and a high concentration of a target sequence-specific reverse transcriptase primer under conditions suitable to generate cDNA; (b) subsequently adding suitable polymerase chain reaction (PCR) reagents to the reverse transcriptase reaction, including a high concentration of a PCR primer set specific to the cDNA and a thermostable DNA polymerase to the reverse transcriptase reaction; and (c) cycling the PCR reaction for a desired number of cycles and under suitable conditions to generate PCR products (“amplicons”) specific to the cDNA. The products of the QRT-PCR process may be compared after a fixed number of PCR cycles to determine the relative quantity of the RNA species as compared to a given reference gene, for example, GAPDH (glyceraldehyde-3-phosphate dehydrogenase). More typically, the progress of the PCR reaction is monitored by analyzing the relative rates of amplicon production for each PCR primer set, for example, by (1) non-specific fluorescent dyes that intercalate with any double-stranded DNA, and/or (2) sequence-specific DNA probes consisting of oligonucleotides that are labeled with a fluorescent reporter which permits detection only after hybridization of the probe with its complementary DNA target.

In some embodiments, gene expression levels may be assessed with a gene array (i.e., gene microarray, gene panel, gene chip). When a gene array is used to assess gene expression levels, complementary nucleic acids (e.g., cDNA, cRNA) that are synthesized from extracted nucleic acids, are contacted with a solid phase having immobilized thereon a plurality of oligonucleotides (e.g., primers, probes) that hybridize specifically to nucleic acids corresponding to or complementary to two or more genes from Table 1. Generally, the oligonucleotides are affixed via a linking chemistry to a solid substrate, such as glass or silicon. Primers and probes for detecting the genes identified in Table 1 may be designed by any routine methods in the art, and may be polynucleotides of any suitable length for detecting any of the two or more genes from Table 1, and may be less than 10, less than 15, less than 20, less than 30, less than 40, less than 50, less than 75, less than 100, less than 200, less than 500, or more than 500 nucleotides in length. The hybridization may then be detected and quantified by methods known in the art (e.g., using fluorophore-labelled cDNA or cRNA) to quantify gene expression levels.

Other suitable non-limiting methods for measuring gene expression include RNA in situ hybridization, RNAse protection assays, Northern blot assays, serial analysis of gene expression (SAGE analysis), immunohistochemistry assays, competitive-binding assays, gene expression analysis by massively parallel signature sequencing (MPSS), etc.

In some embodiments, expression of proteins or polypeptides produced by two or more genes identified in Table 1 may be predictive of whether a candidate substance will be suitable for treating the aesthetic appearance of skin. Any routine methods for assessing protein or polypeptide expression may be used, such as those that utilize antibodies, including flow cytometry, immunohistochemistry, ELISA, Western blot, Northwestern blot, and immunoaffinity chromatograpy. Antibodies may be monoclonal, polyclonal, or any antibody fragment including an Fab, F(ab)₂, Fv, scFv, phage display antibody, peptibody, multispecific ligand, or any other reagent with specific binding to a target. Other suitable methods for assessing protein expression include HPLC, mass spectrometry, protein microarray analysis, PAGE analysis, isoelectric focusing, 2-D gel electrophoresis, and enzymatic assays.

Gene expression levels (or protein expression levels) obtained from skin cells treated with a candidate substance may be compared to gene expression levels from otherwise identical cells treated with a vehicle, in the absence of the candidate substance (control) to determine the relative degree of modulation of the genes, which may comprise upregulation or downregulation of those genes. In some embodiments, the candidate substance will result in upregulation or downregulation of gene expression that is about a 1.3-fold change, about a 1.5-fold change, about a 2-fold change, about a 3-fold change, about a 4-fold change, about a 5-fold change, about a 6-fold change, about a 7-fold change, about an 8-fold change, about a 9-fold change, about a 10-fold change, about a 15-fold change, or about a 20-fold change, relative to control gene expression. Candidate substances meeting these criteria may be selected for use of for further evaluation.

In some embodiments, the expression levels of at least 3 genes, at least 4 genes, at least 5 genes, at least 6 genes, at least 7 genes, at least 8 genes, at least 9 genes, at least 10 genes, at least 15 genes, or at least 20 genes from Table 1 is modulated in a skin cell, following contact with a candidate substance, relative to control.

In some embodiments, the expression levels of at least one gene, at least 2 genes, at least 3 genes, at least 4 genes, or at least 5 genes selected from Table 1 is upregulated in a skin cell, following contact with a candidate substance, relative to control. In another embodiment, the expression levels of at least one gene, at least 2 genes, at least 3 genes, at least 4 genes, or at least 5 genes from Table 1 is downregulated in a skin cell, following contact with a candidate substance, relative to control. In another embodiment, the expression levels of at least one gene from Table 1 is upregulated and the expression levels of at least one gene is selected from Table 1 is downregulated in a skin cell, following contact with a candidate substance, relative to control.

In some embodiments, at least one gene encodes for a cytoplasmic protein. In other embodiments, at least one gene encodes for a nuclear protein. In other embodiments, at least one gene encodes for an extracellular space protein. In other embodiments, at least one gene encodes for a plasma membrane protein

In one embodiment, expression levels of at least one of the genes modulated in a skin cell following contact with a candidate substance, relative to control is selected from the group consisting of: sterile alpha motif domain containing 9; UDP glucuronosyltransferase 2 family, polypeptide A1, complex locus; cytohesin 1 interacting protein; interferon-induced protein 44; interferon-induced protein 44-like; ecotropic viral integration site 2B; G protein-coupled receptor 174; secreted phosphoprotein 1; GLI pathogenesis-related 1; protein tyrosine phosphatase, receptor type, C; and placenta-specific 8.

In one embodiment, expression levels of at least one of the genes modulated in a skin cell following contact with a candidate substance, relative to control is selected from the group consisting of: collagen, type XIV, alpha 1; collagen, type V, alpha 2; collagen, type III, alpha 1; collagen, type VI, alpha 6; and fibroblast activation protein, alpha.

In another embodiment, expression levels of at least secreted phosphoprotein 1 is modulated in a skin cell following contact with a candidate substance, relative to control.

In some embodiments, expression levels of at least one gene involved in extracellular matrix formation, DNA protection and repair, neural regeneration and development, intracellular transport, or actin filament formation, is modulated following contact with a candidate substance, relative to control.

In some embodiments, expression levels are measured of at least two genes other than collagen, type V, alpha 2; collagen, type XIV, alpha 1; collagen, type III, alpha 1; collagen type VI, alpha 6; and/or fibroblast activation protein, alpha.

In other embodiments, expression levels are measured of at least two genes in addition to collagen, type V, alpha 2; collagen, type XIV, alpha 1; collagen, type III, alpha 1; collagen type VI, alpha 6; and/or fibroblast activation protein, alpha.

The invention also provides a gene array (i.e., gene microarray, gene panel, gene chip, biochip) for screening for cosmetic active agents. The gene array may comprise a solid phase having immobilized thereon a plurality of oligonucleotides (e.g., primers, probes, cDNA) that hybridize specifically to nucleic acids corresponding to or complementary to two or more genes from Table 1. Generally, the oligonucleotides are affixed or attached via a linking chemistry to a solid substrate (e.g., glass, silicon, bead, nylon membrane, etc.). The oligonucleotides may be affixed to the solid substrate so that the attachment is stable under conditions of binding, washing, analysis, and removal. In one embodiment, the gene array comprises oligonucleotides that hybridize with nucleic acids from at least 2, at least 3, at least 4, at least 5, at least 6, at least 7, at least 8, at least 9, at least 10, at least 15, at least 20, at least 30, at least 40, or at least 50 of the genes listed in Table 1. In one embodiment, the gene array comprises oligonucleotides that hybridize with nucleic acids corresponding to or complementary to all of the genes listed in Table 1.

In some embodiments, a gene array is provided wherein at least 50%, at least 60%, at least 70%, at least 80%, at least 85%, at least 90%, at least 95%, or at least 99% of the nucleic acids on the gene panel comprise oligonucleotides (e.g., primers, probes) that hybridize with nucleic acids corresponding to or complementary to at least one of the genes listed in Table 1.

Oligonucleotides (e.g., primers, probes) affixed to the solid substrate of the gene array for detecting the genes identified in Table 1 may be designed by any routine methods in the art, and may be polynucleotides of any suitable length for detecting any of the two or more genes from Table 1. Such primers or probes may be single-stranded or double-stranded, and may be less than 10, less than 15, less than 20, less than 30, less than 40, less than 50, less than 75, less than 100, less than 200, less than 500, or more than 500 nucleotides in length. The hybridization may then be detected and quantified by methods known in the art (e.g., using fluorophore-labelled cDNA or cRNA) to quantify gene expression levels.

Compositions are also provided, comprising an effective amount of one or more active ingredients (e.g., chemical compounds, botanical extract, antibody, a nucleic acid (e.g., siRNA), small molecule, etc.) identified by the screening methods of the invention, or identified using the gene arrays of the invention. Cosmetic compositions may be formulated with other cosmetically acceptable, dermatologically acceptable, and/or physiologically acceptable components, such as a vehicle, for topical application to the skin.

The compositions according to the invention can be formulated in a variety of forms for topical application and may comprise from about 0.00001% by weight to about 90% by weight of one or more of the active ingredients identified by the screening methods of the invention. Typically the cosmetic compositions will comprise from about 0.0001% by weight to about 25% by weight, and more preferably from about 0.001% by weight to about 1% by weight of the active ingredient. In one embodiment, the active will comprise from about 0.01% by weight to about 0.1% by weight or to 0.5% by weight of the cosmetic composition. In another embodiment, the active will comprise from about 0.001% by weight to about 5% by weight of the composition. The compositions will comprise an effective amount of one or more of the actives, by which is meant an amount sufficient to treat, prevent, ameliorate, forestall, and/or reduce one or more signs of skin aging or otherwise improve the aesthetic appearance of human skin, in a given area of skin when topically applied thereto.

The cosmetic composition may be formulated in a variety of product forms, such as, for example, a lotion, cream, serum, spray, aerosol, cake, ointment, essence, gel, paste, patch, pencil, towelette, mask, stick, foam, elixir, concentrate, and the like, particularly for topical administration.

Cosmetically or dermatologically acceptable vehicles may include water; vegetable oils; mineral oils; esters such as octal palmitate, isopropyl myristate and isopropyl palmitate; ethers such as dicapryl ether and dimethyl isosorbide; alcohols such as ethanol and isopropanol; fatty alcohols such as cetyl alcohol, cetearyl alcohol, stearyl alcohol and biphenyl alcohol; isoparaffins such as isooctane, isododecane and is hexadecane; silicone oils such as cyclomethicone, dimethicone, dimethicone cross-polymer, polysiloxanes and their derivatives, preferably organomodified derivatives; hydrocarbon oils such as mineral oil, petrolatum, isoeicosane and polyisobutene; polyols such as propylene glycol, glycerin, butylene glycol, pentylene glycol and hexylene glycol; waxes such as beeswax and botanical waxes; or any combinations or mixtures of the foregoing. Aqueous vehicles may include one or more solvents miscible with water, including lower alcohols, such as ethanol, isopropanol, and the like.

The cosmetically acceptable vehicle may be in the form of an emulsion. Non-limiting examples of suitable emulsions include water-in-oil emulsions, oil-in-water emulsions, silicone-in-water emulsions, water-in-silicone emulsions, wax-in-water emulsions, water-oil-water triple emulsions or the like having the appearance of a cream, gel or microemulsions. The emulsion may include an emulsifier, such as a nonionic, anionic or amphoteric surfactant, typically in an amount from about 0.001% to about 5% by weight.

The compositions of the invention are typically suitable for topical application to the human integumentary system, including without limitations skin, nails, hair, etc. The site of application to skin may be skin of the face, lips, hands, chest, etc.

In some embodiments, the compositions may include additional skin actives such as, but are not limited to, botanicals, other keratolytic agents, desquamating agents, keratinocyte proliferation enhancers, collagenase inhibitors, elastase inhibitors, depigmenting agents, anti-inflammatory agents, steroids, anti-acne agents, antioxidants, thiodipropionic acid or esters thereof, and advanced glycation end-product (AGE) inhibitors. Exemplary anti-aging components include, without limitation, botanicals (e.g., Butea Frondosa extract); thiodipropionic acid (TDPA) and esters thereof; retinoids (e.g., all-trans retinoic acid, 9-cis retinoic acid, phytanic acid and others); hydroxy acids (including alpha-hydroxyacids and beta-hydroxyacids), salicylic acid and salicylates; other exfoliating agents (e.g., glycolic acid, 3,6,9-trioxaundecanedioic acid, etc.); estrogen synthetase stimulating compounds (e.g., caffeine and derivatives); compounds capable of inhibiting 5 alpha-reductase activity (e.g., linolenic acid, linoleic acid, finasteride, and mixtures thereof); barrier function enhancing agents (e.g., ceramides, glycerides, cholesterol and its esters, alpha-hydroxy and omega-hydroxy fatty acids and esters thereof, etc.); collagenase inhibitors; and elastase inhibitors; to name a few. In one embodiment, the composition comprises N-Acetyl Tyrosinamide.

Exemplary retinoids include, without limitation, retinoic acid (e.g., all-trans or 13-cis), and derivatives thereof, retinaldehyde, retinol (Vitamin A) and esters thereof, such as retinol palmitate, retinol acetate and retinol propionate, and salts thereof. Particular mention may be made of retinol. It is contemplated that combinations of the compounds of Formulas (I) or (Ia) with any of these retinoids will provide enhanced or synergistic improvements to skin. The retinoids will typically be included in amounts from about 0.0001% to about 5% by weight, more typically from about 0.01% to about 2.5% by weight or from about 0.1% to about 1.0% by weight. Compositions according to this embodiment will typically include an antioxidant such as ascorbic acid and/or BHT and/or a chelating agent such as EDTA or a salt thereof.

In another embodiment, the topical compositions of the present invention may also include one or more of the following: a skin penetration enhancer, an emollient, a humectant, a skin plumper, an optical diffuser, a sunscreen, an additional exfoliating agent, an antioxidant, and a pH adjuster.

An emollient provides the functional benefits of enhancing skin smoothness and reducing the appearance of fine lines and coarse wrinkles Examples include isopropyl myristate, petrolatum, isopropyl lanolate, silicones (e.g., methicone, dimethicone), oils, mineral oils, fatty acid esters, or any mixtures thereof. The emollient may be preferably present from about 0.1 wt % to about 50 wt % of the total weight of the composition.

A skin plumper serves as a collagen enhancer to the skin. An example of a suitable, and preferred, skin plumper is palmitoyl oligopeptide. Other skin plumpers are collagen and/or other glycosaminoglycan (GAG) enhancing agents. When present, the skin plumper may comprise from about 0.1 wt % to about 20 wt % of the total weight of the composition.

A sunscreen for protecting the skin from damaging ultraviolet rays may also be included. Sunscreens include those with a broad range of UVB and UVA protection, such as octocrylene, avobenzone (Parsol 1789), octyl methoxycinnamate, octyl salicylate, oxybenzone, homosylate, benzophenone, camphor derivatives, zinc oxide, and titanium dioxide. When present, the sunscreen may comprise from about 0.01 wt % to about 70 wt % of the composition.

Suitable exfoliating agents include, for example, alpha-hydroxyacids, beta-hydroxyacids, oxaacids, oxadiacids, and their derivatives such as esters, anhydrides and salts thereof. Suitable hydroxy acids include, for example, glycolic acid, lactic acid, malic acid, tartaric acid, citric acid, 2-hydroxyalkanoic acid, mandelic acid, salicylic acid and other derivatives thereof. A preferred exfoliating agent is glycolic acid. When present, the exfoliating agent may comprise from about 0.1 wt % to about 80 wt % of the composition.

An antioxidant functions, among other things, to scavenge free radicals from skin to protect the skin from environmental aggressors. Examples of antioxidants that may be used in the present compositions include compounds having phenolic hydroxy functions, such as ascorbic acid and its derivatives/esters; beta-carotene; catechins; curcumin; ferulic acid derivatives (e.g., ethyl ferulate, sodium ferulate); gallic acid derivatives (e.g., propyl gallate); lycopene; reductic acid; rosmarinic acid; tannic acid; tetrahydrocurcumin; tocopherol and its derivatives; uric acid; or any mixtures thereof. Other suitable antioxidants are those that have one or more thiol functions (—SH), in either reduced or non-reduced form, such as glutathione, lipoic acid, thioglycolic acid, and other sulfhydryl compounds. The antioxidant may be inorganic, such as bisulfites, metabisulfites, sulfites, or other inorganic salts and acids containing sulfur. In one particular embodiment, the inventive compositions will include TDPA or an ester thereof (e.g., dilauryl thiodipropionic acid), and/or an alpha hydroxyl acid (glycolic acid) and/or beta hydroxyl acid (salicylic acid or a derivative). Compositions of the present invention may comprise an antioxidant, which may comprise from about 0.001 wt % to about 10 wt %, or from about 0.01 wt % to about 5 wt %, of the total weight of the composition.

Other conventional additives include: vitamins, such as tocopherol and ascorbic acid; vitamin derivatives such as ascorbyl monopalmitate; thickeners such as hydroxyalkyl cellulose; gelling agents; structuring agents; metal chelating agents such as EDTA or salts thereof; pigments; colorants; and pH adjusters. The composition may optionally comprise other components known to those skilled in the art including, but not limited to, film formers, moisturizers, minerals, viscosity and/or rheology modifiers, anti-acne agents, insect repellents, skin cooling compounds, skin protectants, lubricants, fragrances, preservatives, stabilizers, and mixtures thereof. In addition to the foregoing, the cosmetic compositions of the invention may contain any other compound for the treatment of skin disorders. The conventional additives, actives, adjuvants, and excipients set forth in the preceding paragraphs are present in the compositions in amounts suitable to obtain their intended purpose and effect, each typically being present in an amount of from 0.01 to 25% by weight of the cosmetic composition, in particular from about 0.1 to 5% by weight of the cosmetic composition.

The compositions may include liposomes. The liposomes may comprise other additives or substances and/or may be modified to more specifically reach or remain at a site following administration.

In one embodiment, the composition of the invention may have a pH between about 1 and about 8. In certain embodiments, the pH of the composition will be acidic, i.e., less than 7.0, and preferably will be between about 2 and about 7, more preferably between about 3.5 and about 5.5.

The compositions may be applied to the skin for a period of time sufficient to diminish the appearance of melanin in the skin. The compositions may be applied topically once, twice, or more daily. The treatment may be for a period of one week, two weeks, four weeks, eight weeks, or more.

In some embodiments, the invention provides methods for improving the aesthetic appearance of skin, reducing or treating one or more signs of skin aging, and/or treating aging skin by topically applying a cosmetic composition identified by the screening methods of the invention, preferably in a cosmetically acceptable vehicle, over the affected area for a period of time sufficient to reduce, ameliorate, reverse or prevent dermatological signs of aging. This method is particularly useful for treating signs of skin photoaging and intrinsic aging.

Generally, the improvement in the condition and/or aesthetic appearance is selected from the group consisting of: reducing dermatological signs of chronological aging, photo-aging, hormonal aging, and/or actinic aging; preventing and/or reducing the appearance of lines and/or wrinkles; reducing the noticeability of facial lines and wrinkles, facial wrinkles on the cheeks, forehead, perpendicular wrinkles between the eyes, horizontal wrinkles above the eyes, and around the mouth, marionette lines, and particularly deep wrinkles or creases; preventing, reducing, and/or diminishing the appearance and/or depth of lines and/or wrinkles; improving the appearance of suborbital lines and/or periorbital lines; reducing the appearance of crow's feet; rejuvenating and/or revitalizing skin, particularly aging skin; reducing skin fragility; preventing and/or reversing of loss of glycosaminoglycans and/or collagen; ameliorating the effects of estrogen imbalance; preventing skin atrophy; preventing, reducing, and/or treating hyperpigmentation; minimizing skin discoloration; improving skin tone, radiance, clarity and/or tautness; preventing, reducing, and/or ameliorating skin sagging; improving skin firmness, plumpness, suppleness and/or softness; improving procollagen and/or collagen production; improving skin texture and/or promoting retexturization; improving skin barrier repair and/or function; improving the appearance of skin contours; restoring skin luster and/or brightness; minimizing dermatological signs of fatigue and/or stress; resisting environmental stress; replenishing ingredients in the skin decreased by aging and/or menopause; improving communication among skin cells; increasing cell proliferation and/or multiplication; increasing skin cell metabolism decreased by aging and/or menopause; retarding cellular aging; improving skin moisturization; enhancing skin thickness; increasing skin elasticity and/or resiliency; enhancing exfoliation; improving microcirculation; decreasing and/or preventing cellulite formation; and any combinations thereof.

The cosmetic composition will typically be applied to the skin at least one, at least two, or at least three times daily for as long as is necessary to achieve desired results (e.g., anti-aging results or improvement in the health and/or appearance of the skin). The treatment regimen may comprise at least daily application for at least one week, at least two weeks, at least four weeks, at least eight weeks, or at least twelve weeks. Chronic treatment regimens are also contemplated.

The aesthetic improvement in the appearance of human skin (i e, improvement in one or more dermatological signs of aging) achieved with the compounds identified according to the screening methods of the invention may include, without limitation, one or more of the following:
(a) treatment, reduction, and/or prevention of fine lines or wrinkles;
(b) reduction of skin pore size;
(c) improvement in skin thickness, plumpness, and/or tautness;
(d) improvement in skin smoothness, suppleness and/or softness;
(e) improvement in skin tone, radiance, and/or clarity;
(f) improvement in procollagen, and/or collagen production;
(g) improvement in maintenance and remodeling of elastin;
(h) improvement in skin texture and/or promotion of retexturization;
(i) improvement in skin barrier repair and/or function;
(j) improvement in appearance of skin contours;
(k) restoration of skin luster and/or brightness;
(l) replenishment of essential nutrients and/or constituents in the skin;
(m) improvement of skin appearance decreased by aging and/or menopause;
(n) improvement in skin moisturization;
(o) increase in skin elasticity and/or resiliency;
(p) treatment, reduction, and/or prevention of skin sagging;
(q) improvement in skin firmness;
(r) reduction of pigment spots and/or mottled skin; and
(s) improvement of optical properties of skin by light diffraction or reflection.

In practice, the compositions of the invention are applied to skin in need of treatment. Skin in need of treatment typically includes skin that suffers from a deficiency or loss in any of the foregoing attributes or which would otherwise benefit from improvement in any of the foregoing skin attributes.

In certain embodiments the compositions and methods are provided for the prevention, treatment, and/or amelioration of fine lines and/or wrinkles in the skin. In this case, the compositions are applied to skin in need of treatment, by which is meant skin having wrinkles and/or fine lines. In some embodiments, the compositions are applied directly to the fine lines and/or wrinkles. The compositions and methods are suitable for treating fine lines and/or wrinkles on any surface of the skin, including without limitation, the skin of the face, neck, and/or hands.

In other embodiments, the compositions and methods of the invention are directed to the prevention, treatment, and/or amelioration of blemishes, acne, hyperpigmentation, or hypopigmentation in human skin. In this case, the compositions are applied to skin in need of treatment, by which is meant skin having a blemish, acne, hyperpigmentation, or hypopigmentation. The compositions may be applied directly to the blemish, acne, or area of the skin that is hyperpigmented (e.g., age spots or freckles) or hypopigmented (e.g., pale patch).

In other preferred embodiments, the compositions and methods of the invention are directed to promoting exfoliation of human skin. In this case, the compositions are applied to skin in need of treatment, by which is meant skin in need of exfoliation. The compositions may be applied directly to the area of skin in need of exfoliation.

In other embodiments, the compositions and methods of the invention may be used to treat, prevent, or ameliorate skin pigmentation, dandruff, seborrheic dermatitis, ringworm infection, psoriasis, calluses, ichthyosis, and warts.

The compositions are topically applied to an “individual in need thereof,” by which is meant an individual that stands to benefits from reducing visible signs of skin damage or aging. In a specific embodiment, the active ingredient is provided in a pharmaceutically, physiologically, cosmetically, and/or dermatologically-acceptable vehicle, diluent, or carrier, where the composition is topically applied to an affected area of skin and left to remain on the affected area in an amount effective for improving the condition and aesthetic appearance of skin.

In one embodiment, methods of treating skin are provided, comprising topically administering to skin in need thereof an effective amount of a composition of the invention (i.e., comprising an active agent identified by the screening methods of the invention or identified by the gene arrays of the invention) for a time sufficient to improve the aesthetic appearance of skin or one or more dermatological signs of aging.

In one embodiment, methods for treating and improving the signs of skin aging (e.g., fine lines and wrinkles), methods for reducing blemishes or acne, and methods for promoting exfoliation of skin comprise topically applying a composition of the invention to the skin of an individual in need thereof (e.g., topical application directly to the fine line and/or wrinkle, to the blemish or acne, to the area of skin in need of exfoliation) in an amount and for a time sufficient to reduce the severity of the fine lines and/or wrinkles, to reduce the blemish or acne, or to promote exfoliation, or to prevent or inhibit the formation of new fine lines and/or wrinkles, the formation of acne or a blemish, or to prevent the need for additional exfoliation.

The effect of a composition on the formation or appearance of fine lines and wrinkles, of a blemish, of acne, of age spots, etc., can be evaluated qualitatively, e.g., by visual inspection, or quantitatively, e.g., by microscopic or computer assisted measurements of wrinkle morphology (e.g., the number, depth, length, area, volume and/or width of wrinkles per unit area of skin), etc.

It is also contemplated that the compositions of the invention will be useful for treating thin skin by topically applying the composition to thin skin of an individual in need thereof “Thin skin” is intended to include skin that is thinned due to chronological aging, menopause, or photo-damage. In some embodiments, the treatment is for thin skin in men, whereas other embodiments treat thin skin in women, pre-menopausal or post-menopausal, as it is believed that skin thins differently with age in men and women, and in particular in women at different stages of life.

The methods of the invention may be employed prophylactically to forestall aging including in patients that have not manifested signs of skin aging, most commonly in male or female individuals under 25 years of age, under 35 years of age, or under 45 years of age. The methods may also reverse or treat signs of aging once manifested as is common in male or female individuals over 25 years of age, over 35 years of age, over 45 years of age, over 55 years of age, or over 65 years of age. The methods of the invention may also be used in male or female individuals either under 25 years of age or 25 years of age or older, to prevent, reverse, or treat wrinkles and/or fine lines, thick skin, acne, blemishes, hyperpigmentation, to improve the aesthetic appearance of skin, or to promote exfoliation of skin.

The following examples are meant to demonstrate certain aspects of the invention in a non-limiting fashion.

### EXAMPLES

### Example 1

In order to identify a novel set of genes that can be used to screen for active agents for treating skin, gene expression was measured in untreated skin, and compared with gene expression in skin that had been subject to one of three well-tested, standard skin therapies. Specifically, gene array analyses were conducted on mRNA isolated from human skin following exposure of the skin to: laser therapy (“Laser Group”), retinoic acid treatment (“RA Group”), or retinol (“Retinol Group”).

The Laser Group (n=6) included subjects over the age of 18 that were determined to have photodamaged skin following a cutaneous physical examination of the forearm and pre-auricular area. Baseline biopsies were obtained from the forearm (up to 4 mm in diameter) or from the pre-auricular area of the face (up to 2 mm in diameter) of subjects. A portion of each subject's forearm and/or pre-auricular skin was then treated a single time using the Ultrapulse® Encore™ fractionated carbon dioxide laser at laser settings within the range of those typically used in a clinical setting. Biopsies were taken from the treated skin at 48 hours and at 3 weeks following laser treatment. Collected tissue was fixed in RNAlater and then analyzed for gene expression.

The Retinoic Acid Group (n=15) included subjects over the age of 18 that were determined to have photodamaged skin on the dorsal aspect of the forearm. Baseline biopsies (4 mm) were taken from the dorsal aspect of the forearm. The dorsal aspect of the forearm was then treated with 0.02% retinoic acid in the form of a partially occlusive patch for 30 days. Additional biopsies (4 mm) were obtained 4 days, 8 days, 15 days, and 30 days following treatment. Collected tissue was fixed in RNAlater and then analyzed for gene expression.

The Retinol Group (n=16) included subjects over the age of 18 that were determined to have photodamaged skin on the dorsal aspect of the forearm. Baseline biopsies (4 mm) were taken from the dorsal aspect of the forearm. The dorsal aspect of the forearm was then treated with a 0.02% retinol patch in the form of a partially occlusive patch for 30 days. Additional biopsies (4 mm) were obtained 4 days, 8 days, 15 days, and 30 days following treatment. Collected tissue was fixed in RNAlater and then analyzed for gene expression.

RNA was extracted from all tissue biopsy samples using the RNeasy method (Qiagen). Briefly, tissue was homogenized and lysed in RLT buffer (with beta-mercaptoethanol) using Lysing Matrix D tubes (MP Biomedicals LLC). Homogenates were transferred to collection tubes which were then loaded into a QIAcube automated sample prep instrument to perform the remainder of the RNeasy extraction. The resulting RNA was quantitated by spectrophotometry using a NanoDrop ND-8000 (Thermo), and was evaluated for integrity using the RNA 6000 Nano Assay on a Bioanalyzer 2100 (Agilent). Purified RNA samples were stored at −80° C.

RNA samples were converted into labelled target antisense RNA (cRNA) using the Single-Round RNA Amplification and Biotin Labelling System (Enzo Biochem). Briefly, 1 μg of total RNA was converted into double stranded cDNA via reverse transcription using an oligo-d(T) primer-adaptor. This cDNA was purified and used as a template for in vitro transcription using T7 RNA polymerase and biotinylated ribonucleotides. The resulting cRNA was purified using magnetic beads and quantitated using spectrophotometry. Next, 11 μg of purified cRNA was fragmented using a 5× Fragmentation buffer, and then a hybridization cocktail was prepared and added to the fragmentation product using the Hybridization, Wash and Stain kit (Affymetrix). This was applied to Human U133 Plus 2.0 gene arrays, and incubated at 45° C. for 16 hours. Following hybridization, arrays were washed and stained using standard Affymetrix procedures before scanning on the Affymetrix GeneChip Scanner. Data were extracted using Expression Console.

Gene expression was examined to determine which genes showed at least a 1.3 fold change in expression (whether upregulated or downregulated) after each of the three treatments (laser, RA, and retinol), relative to expression of those genes in skin prior to treatment (at baseline). In the Laser Group, gene expression levels from the 3-week time point was used for gene expression analysis. In the RA Group and Retinol Group, gene expression levels from the 30-day time point was used for gene expression analysis. A unique set of 59 genes demonstrated at least a 1.3 fold change in expression levels following each of the three treatments relative to baseline. These genes, their symbols, names, and their associated accession numbers (according to the National Center for Biotechnology Information (NCBI) database) are identified below in Table 1, along with the location of the expressed protein, and the fold-change quantified for each group. A negative number indicates that the gene was downregulated relative to baseline, and a positive number indicates that the gene was upregulated relative to baseline.

TABLE 1

| | NCBI | | | Laser | RA | Retinol |
|---|---|---|---|---|---|---|
| NCBI | RefSeq | NCBI Entrez | | (fold | (fold | (fold |
| Symbol | Accession No. | Gene Name | Location | change) | change) | change) |
| UGT2A1 | NM_006798 | Homo sapiens | Cytoplasm | 3.255 | −1.369 | −1.696 |
| | | UDP glucurono- | | | | |
| | | syltransferase 2 | | | | |
| | | family, | | | | |
| | | polypeptide A1, | | | | |
| | | complex locus, | | | | |
| | | transcript variant 1, | | | | |
| | | mRNA | | | | |
| PCSK2 | NM_002594 | Homo sapiens | Extracellular | −1.513 | −1.825 | −1.566 |
| | | proprotein convertase | space | | | |
| | | subtilisin/kexin type 2, | | | | |
| | | transcript variant 1, | | | | |
| | | mRNA | | | | |
| TRPM1 | NM_002420 | Homo sapiens | Plasma | −1.315 | −1.306 | −1.431 |
| | | transient receptor | membrane | | | |
| | | potential cation | | | | |
| | | channel, subfamily M, | | | | |
| | | member 1, transcript | | | | |
| | | variant 2, mRNA | | | | |
| CHRM4 | NM_000741 | Homo sapiens | Plasma | −1.336 | −1.326 | −1.401 |
| | | cholinergic receptor, | membrane | | | |
| | | muscarinic 4, mRNA | | | | |
| DIO2 | NM_013989 | Homo sapiens | Cytoplasm | 2.149 | −1.548 | −1.384 |
| | | deiodinase, | | | | |
| | | iodothyronine, | | | | |
| | | type II, transcript | | | | |
| | | variant 1, mRNA | | | | |
| GDPD2 | NM_017711 | Homo sapiens | Plasma | −1.459 | −2.585 | −1.363 |
| | | glycerophosphodiester | membrane | | | |
| | | phosphodiesterase | | | | |
| | | domain | | | | |
| | | containing 2, | | | | |
| | | transcript variant 2, | | | | |
| | | mRNA | | | | |
| HIST1H3A | NM_003529 | Homo sapiens | Nucleus | 1.550 | 1.375 | −1.363 |
| | | histone cluster | | | | |
| | | 1, H3a, mRNA | | | | |
| OCA2 | NM_000275 | Homo sapiens | Plasma | −1.379 | −1.432 | −1.334 |
| | | oculocutaneous | membrane | | | |
| | | albinism II, | | | | |
| | | transcript variant 1, | | | | |
| | | mRNA | | | | |
| TPPP | NM_007030 | Homo sapiens | Cytoplasm | −1.441 | −1.402 | −1.328 |
| | | tubulin | | | | |
| | | polymerization | | | | |
| | | promoting | | | | |
| | | protein, mRNA | | | | |
| GMFG | NM_004877 | Homo sapiens | Cytoplasm | 1.865 | 1.358 | 1.300 |
| | | glia maturation | | | | |
| | | factor, gamma, | | | | |
| | | transcript variant 1, | | | | |
| | | mRNA | | | | |
| C1orf162 | NM_174896 | Homo sapiens | Unknown | 1.897 | 1.356 | 1.301 |
| | | chromosome 1 | | | | |
| | | open reading | | | | |
| | | frame 162, | | | | |
| | | transcript variant 1, | | | | |
| | | mRNA | | | | |
| SELL | NM_000655 | Homo sapiens | Plasma | 1.813 | 1.691 | 1.304 |
| | | selectin L, | membrane | | | |
| | | transcript variant 1, | | | | |
| | | mRNA | | | | |
| CYP7B1 | NM_004820 | Homo sapiens | Cytoplasm | 2.093 | 1.320 | 1.309 |
| | | cytochrome | | | | |
| | | P450, family 7, | | | | |
| | | subfamily B, | | | | |
| | | polypeptide 1, | | | | |
| | | mRNA | | | | |
| CD2 | NM_001767 | Homo sapiens | Plasma | 1.311 | 1.339 | 1.311 |
| | | CD2 molecule, | membrane | | | |
| | | mRNA | | | | |
| IFI44L | NM_006820 | Homo sapiens | Cytoplasm | 3.835 | 1.472 | 1.320 |
| | | interferon- | | | | |
| | | induced protein | | | | |
| | | 44-like, mRNA | | | | |
| FPR3 | NM_002030 | Homo sapiens | Plasma | 2.226 | 1.439 | 1.321 |
| | | formyl peptide | membrane | | | |
| | | receptor 3, mRNA | | | | |
| EVI2B | NM_006495 | Homo sapiens | Plasma | 2.951 | 1.405 | 1.323 |
| | | ecotropic viral | membrane | | | |
| | | integration site | | | | |
| | | 2B, mRNA | | | | |
| GPR174 | NM_032553 | Homo sapiens | Plasma | 2.862 | 1.411 | 1.339 |
| | | G protein-coupled | membrane | | | |
| | | receptor 174, mRNA | | | | |
| FCGR2A | NM_001136219 | Homo sapiens | Plasma | 1.658 | 1.366 | 1.346 |
| | | Fc fragment of | membrane | | | |
| | | IgG, low | | | | |
| | | affinity IIa, | | | | |
| | | receptor (CD32), | | | | |
| | | transcript variant 1, | | | | |
| | | mRNA | | | | |
| CD86 | NM_175862 | Homo sapiens | Plasma | 1.887 | 1.468 | 1.348 |
| | | CD86 molecule, | membrane | | | |
| | | transcript | | | | |
| | | variant 1, mRNA | | | | |
| SRGN | NM_002727 | Homo sapiens | Extracellular | 2.656 | 1.569 | 1.353 |
| | | serglycin, transcript | space | | | |
| | | variant 1, mRNA | | | | |
| PARP14 | NM_017554 | Homo sapiens | Cytoplasm | 2.133 | 1.364 | 1.356 |
| | | poly (ADP-ribose) | | | | |
| | | polymerase | | | | |
| | | family, member | | | | |
| | | 14, mRNA | | | | |
| VCAM1 | NM_001078 | Homo sapiens | Plasma | 1.777 | 1.730 | 1.357 |
| | | vascular cell | membrane | | | |
| | | adhesion molecule 1, | | | | |
| | | transcript variant 1, | | | | |
| | | mRNA | | | | |
| IL33 | NM_033439 | Homo sapiens | Extracellular | 2.808 | 1.587 | 1.360 |
| | | interleukin 33, | space | | | |
| | | transcript variant 1, | | | | |
| | | mRNA | | | | |
| HLA-DPA1 | NM_033554 | Homo sapiens | Plasma | 1.579 | 1.466 | 1.364 |
| | | major | membrane | | | |
| | | histocompatibility | | | | |
| | | complex, class II, | | | | |
| | | DP alpha 1, | | | | |
| | | transcript variant 1, | | | | |
| | | mRNA | | | | |
| ST8SIA4 | NM_005668 | Homo sapiens | Cytoplasm | 2.254 | 1.389 | 1.366 |
| | | ST8 alpha-N-acetyl- | | | | |
| | | neuraminide | | | | |
| | | alpha-2,8- | | | | |
| | | sialyltransferase 4, | | | | |
| | | transcript variant 1, | | | | |
| | | mRNA | | | | |
| MIRLET7C | NR_029480 | Homo sapiens | Cytoplasm | 2.600 | −1.319 | 1.366 |
| | | microRNA let- | | | | |
| | | 7c, microRNA | | | | |
| KIAA0226L | NM_025113 | Homo sapiens | Unknown | 1.507 | 1.307 | 1.371 |
| | | KIAA0226-like, | | | | |
| | | transcript variant 1, | | | | |
| | | mRNA | | | | |
| KLRB1 | NM_002258 | Homo sapiens | Plasma | 2.123 | 1.363 | 1.377 |
| | | killer cell lectin- | membrane | | | |
| | | like receptor | | | | |
| | | subfamily B, | | | | |
| | | member 1, mRNA | | | | |
| HLA-DQA1 | NM_002122 | Homo sapiens | Plasma | 2.108 | 1.594 | 1.378 |
| | | major | membrane | | | |
| | | histocompatibility | | | | |
| | | complex, | | | | |
| | | class II, DQ | | | | |
| | | alpha 1, mRNA | | | | |
| RGS1 | NM_002922 | Homo sapiens | Plasma | 2.462 | 1.503 | 1.379 |
| | | regulator of G- | membrane | | | |
| | | protein signaling 1, | | | | |
| | | mRNA | | | | |
| COL5A2 | NM_000393 | Homo sapiens | Extracellular | 2.012 | 1.369 | 1.380 |
| | | collagen, type | space | | | |
| | | V, alpha 2, | | | | |
| | | mRNA | | | | |
| SAMD9 | NM_017654 | Homo sapiens | Cytoplasm | 2.950 | 5.339 | 1.382 |
| | | sterile alpha | | | | |
| | | motif domain | | | | |
| | | containing 9, | | | | |
| | | transcript variant 1, | | | | |
| | | mRNA | | | | |
| IRF8 | NM_002163 | Homo sapiens | Nucleus | 1.607 | 1.725 | 1.392 |
| | | interferon regulatory | | | | |
| | | factor 8, mRNA | | | | |
| CD226 | NM_006566 | Homo sapiens | Plasma | 2.030 | 1.423 | 1.396 |
| | | CD226 molecule, | membrane | | | |
| | | mRNA | | | | |
| GZMK | NM_002104 | Homo sapiens | Cytoplasm | 2.759 | 1.571 | 1.402 |
| | | granzyme K | | | | |
| | | (granzyme 3; | | | | |
| | | tryptase II), mRNA | | | | |
| FYB | NM_001465 | Homo sapiens | Nucleus | 1.895 | 1.483 | 1.404 |
| | | FYN binding | | | | |
| | | protein, transcript | | | | |
| | | variant 1, mRNA | | | | |
| COL14A1 | NM_021110 | Homo sapiens | Extracellular | 1.702 | 1.311 | 1.408 |
| | | collagen, type | space | | | |
| | | XIV, alpha 1, mRNA | | | | |
| CYBB | NM_000397 | Homo sapiens | Cytoplasm | 2.601 | 1.637 | 1.413 |
| | | cytochrome b- | | | | |
| | | 245, beta | | | | |
| | | polypeptide, mRNA | | | | |
| CCR2 | NM_001123396 | Homo sapiens | Plasma | 1.863 | 1.658 | 1.416 |
| | | chemokine (C-C | membrane | | | |
| | | motif) receptor | | | | |
| | | 2, transcript | | | | |
| | | variant B, mRNA | | | | |
| IFI44 | NM_006417 | Homo sapiens | Unknown | 4.119 | 1.337 | 1.429 |
| | | interferon- | | | | |
| | | induced protein | | | | |
| | | 44, mRNA | | | | |
| PLAC8 | NM_016619 | Homo sapiens | Nucleus | 3.055 | 1.844 | 1.434 |
| | | placenta-specific 8, | | | | |
| | | transcript variant 2, | | | | |
| | | mRNA | | | | |
| HLA-DRA | NM_019111 | Homo sapiens | Plasma | 1.794 | 1.403 | 1.439 |
| | | major | membrane | | | |
| | | histocompatibility | | | | |
| | | complex, | | | | |
| | | class II, DR | | | | |
| | | alpha, mRNA | | | | |
| PTPRC | NM_002838 | Homo sapiens | Plasma | 3.086 | 1.606 | 1.446 |
| | | protein tyrosine | membrane | | | |
| | | phosphatase, | | | | |
| | | receptor type, | | | | |
| | | C, transcript | | | | |
| | | variant 1, mRNA | | | | |
| PLEK | NM_002664 | Homo sapiens | Cytoplasm | 1.975 | 1.667 | 1.449 |
| | | pleckstrin, mRNA | | | | |
| GLIPR1 | NM_006851 | Homo sapiens | Extracellular | 3.410 | 1.422 | 1.450 |
| | | GLI | space | | | |
| | | pathogenesis- | | | | |
| | | related 1, mRNA | | | | |
| RNASE6 | NM_005615 | Homo sapiens | Extracellular | 2.338 | 1.540 | 1.452 |
| | | ribonuclease, | space | | | |
| | | RNase A family, k6, | | | | |
| | | mRNA | | | | |
| COL3A1 | NM_000090 | Homo sapiens | Extracellular | 2.610 | 1.443 | 1.455 |
| | | collagen, type | space | | | |
| | | III, alpha 1, mRNA | | | | |
| CYTIP | NM_004288 | Homo sapiens | Cytoplasm | 2.954 | 1.336 | 1.481 |
| | | cytohesin 1 | | | | |
| | | interacting | | | | |
| | | protein, mRNA | | | | |
| CD53 | NM_000560 | Homo sapiens | Plasma | 2.455 | 1.586 | 1.483 |
| | | CD53 molecule, | membrane | | | |
| | | transcript variant 2, | | | | |
| | | mRNA | | | | |
| CLEC7A | NM_197947 | Homo sapiens | Plasma | 2.324 | 1.964 | 1.502 |
| | | C-type lectin | membrane | | | |
| | | domain family | | | | |
| | | 7, member A, | | | | |
| | | transcript variant 1, | | | | |
| | | mRNA | | | | |
| CTSS | NM_004079 | Homo sapiens | Cytoplasm | 2.178 | 1.302 | 1.514 |
| | | cathepsin S, transcript | | | | |
| | | variant 1, mRNA | | | | |
| FAP | NM_004460 | Homo sapiens | Cytoplasm | 5.031 | 1.370 | 1.518 |
| | | fibroblast activation | | | | |
| | | protein, alpha, | | | | |
| | | transcript variant 1, | | | | |
| | | mRNA | | | | |
| LYZ | NM_000239 | Homo sapiens | Extracellular | 2.762 | 2.271 | 1.603 |
| | | lysozyme, mRNA | space | | | |
| SPP1 | NM_001040058 | Homo sapiens | Extracellular | 12.170 | 1.416 | 1.604 |
| | | secreted | space | | | |
| | | phosphoprotein | | | | |
| | | 1, transcript | | | | |
| | | variant 1, mRNA | | | | |
| MNDA | NM_002432 | Homo sapiens | Nucleus | 1.673 | 1.406 | 1.805 |
| | | myeloid cell nuclear | | | | |
| | | differentiation | | | | |
| | | antigen, mRNA | | | | |
| BIRC3 | NM_001165 | Homo sapiens | Cytoplasm | 2.425 | 1.555 | 1.815 |
| | | baculoviral IAP | | | | |
| | | repeat containing 3, | | | | |
| | | transcript variant 1, | | | | |
| | | mRNA | | | | |
| CD1B | NM_001764 | Homo sapiens | Plasma | 2.025 | 1.510 | 1.844 |
| | | CD1b molecule, | membrane | | | |
| | | mRNA | | | | |
| COL6A6 | NM_001102608 | Homo sapiens | Extracellular | 1.899 | 2.091 | 2.245 |
| | | collagen, type | space | | | |
| | | VI, alpha 6, mRNA | | | | |

It will be understood that in the event of any inconsistency between the gene symbol, accession number, and gene name in Table 1, all such nucleic acids will be considered as embraced by the invention. For example, in the event of a discrepancy in the accession number and the gene name provided, both the gene corresponding to the accession number and the gene corresponding to the gene name will be considered within the scope of the invention.

Following treatment of skin with a candidate substance, modulation of expression of any two or more of the 59 genes identified in Table 1 (or expression of any homolog, fragment or marker of those genes) indicates that the candidate substance may be used as a skin active, for providing a therapeutic benefit to the skin.

All references including patent applications and publications cited herein are incorporated herein by reference in their entirety and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. A method of screening for active agents comprising:
(a) contacting a human skin cell with a candidate substance; and
(b) determining whether expression of at least two genes is modulated in said skin cell, said genes being selected from the group consisting of: UDP glucuronosyltransferase 2 family, polypeptide A1, complex locus; proprotein convertase subtilisin/kexin type 2; transient receptor potential cation channel, subfamily M, member 1; cholinergic receptor, muscarinic 4; deiodinase, iodothyronine, type II; glycerophosphodiester phosphodiesterase domain containing 2; histone cluster 1, H3a; oculocutaneous albinism II; tubulin polymerization promoting protein; glia maturation factor, gamma; chromosome 1 open reading frame 162; selectin L; cytochrome P450, family 7, subfamily B, polypeptide 1; CD2 molecule; interferon-induced protein 44-like; formyl peptide receptor 3; ecotropic viral integration site 2B; G protein-coupled receptor 174; Fc fragment of IgG, low affinity IIa, receptor (CD32); CD86 molecule; Serglycin; poly (ADP-ribose) polymerase family, member 14; vascular cell adhesion molecule 1; interleukin 33; major histocompatibility complex, class II, DP alpha 1; ST8 alpha-N-acetyl-neuraminide alpha-2,8-sialyltransferase 4; microRNA let-7c; KIAA0226-like; killer cell lectin-like receptor subfamily B, member 1; major histocompatibility complex, class II, DQ alpha 1; regulator of G-protein signaling 1; collagen, type V, alpha 2; sterile alpha motif domain containing 9; interferon regulatory factor 8; CD226 molecule; granzyme K (granzyme 3; tryptase II); FYN binding protein; collagen, type XIV, alpha 1; cytochrome b-245, beta polypeptide; chemokine (C-C motif) receptor 2; interferon-induced protein 44; placenta-specific 8; major histocompatibility complex, class II, DR alpha; protein tyrosine phosphatase, receptor type, C; pleckstrin; GLI pathogenesis-related 1; ribonuclease, RNase A family, k6; collagen, type III, alpha 1; cytohesin 1 interacting protein; CD53 molecule; C-type lectin domain family 7, member A; cathepsin S; fibroblast activation protein, alpha; lysozyme; secreted phosphoprotein 1; myeloid cell nuclear differentiation antigen; baculoviral IAP repeat containing 3; CD1b molecule; and collagen, type VI, alpha 6;
wherein modulation of said at least two genes indicates that said candidate substance is an active agent suitable for improving the appearance and/or health of skin.

2. The method according to claim 1, wherein expression of said two or more genes is determined using an array comprising a solid phase having attached thereto a nucleic acid complementary to or corresponding to a least a portion of a sequence of said two or more genes.

3. The method according to claim 2, wherein said skin cell is a fibroblast or a keratinocyte.

4. The method according to claim 2, further comprising extracting nucleic acids from said skin cell.

5. The method according to claim 4, wherein said step of determining further comprises quantifying said nucleic acids extracted from said skin cell.

6. The method according to claim 2, further comprising determining whether expression of said two or more genes is modulated in a skin cell that has not been contacted with said candidate substance.

7. The method according to claim 2, wherein said modulation comprises upregulation or downregulation of said two or more genes.

8. The method according to claim 2, wherein said modulation comprises at least a 1.2-fold change in gene expression relative to expression of the same genes in a skin cell that has not been contacted with said candidate substance.

9. The method according to claim 2, wherein expression of at least three genes is modulated in said skin cell.

10. The method according to claim 2, wherein expression of at least one gene is upregulated and expression of at least one gene is downregulated in said skin cell.

11. The method according to claim 2, wherein at least one of said genes modulated in said skin cell is selected from the group consisting of: sterile alpha motif domain containing 9; UDP glucuronosyltransferase 2 family, polypeptide A1, complex locus; cytohesin 1 interacting protein; interferon-induced protein 44; interferon-induced protein 44-like; ecotropic viral integration site 2B; G protein-coupled receptor 174; secreted phosphoprotein 1; GLI pathogenesis-related 1; protein tyrosine phosphatase, receptor type, C; and placenta-specific 8.

12. The method according to claim 2, wherein at least one of said genes modulated in said skin cell is selected from the group consisting of: collagen, type XIV, alpha 1; collagen, type V, alpha 2; collagen, type III, alpha 1; and collagen, type VI, alpha 6.

13. The method according to claim 2, wherein at least one of said genes modulated in said skin cell is secreted phosphoprotein 1.

14. A cosmetic composition comprising, in a cosmetically acceptable vehicle, one or more active agents identified according to the screening method of claim 2.

15. The cosmetic composition according to claim 13, further comprising a cosmetic ingredient selected from the group consisting of a botanical extract, film forming polymer, a thickener, a pH adjuster, a preservative, an emulsifier, a gelling agent, an antioxidant, an emollient, a humectant, a fragrance, and a colorant.

16. A method for improving the appearance and/or health of human skin comprising topically applying to an area of the skin in need thereof a composition according to claim 14.

17. The method according to claim 16, wherein said improvement in the appearance and/or health of said human skin is selected from the group consisting of:
(a) treatment, reduction, and/or prevention of fine lines or wrinkles;
(b) reduction of skin pore size;
(c) improvement in skin thickness, plumpness, and/or tautness;
(d) improvement in skin smoothness, suppleness and/or softness;
(e) improvement in skin tone, radiance, and/or clarity;
(f) improvement in procollagen, and/or collagen production;
(g) improvement in maintenance and remodeling of elastin;
(h) improvement in skin texture and/or promotion of retexturization;
(i) improvement in skin barrier repair and/or function;
(j) improvement in appearance of skin contours;
(k) restoration of skin luster and/or brightness;
(l) replenishment of essential nutrients and/or constituents in the skin;
(m) improvement of skin appearance decreased by aging and/or menopause;
(n) improvement in skin moisturization;
(o) increase in skin elasticity and/or resiliency;
(p) treatment, reduction, and/or prevention of skin sagging;
(q) improvement in skin firmness;
(r) reduction of pigment spots and/or mottled skin; and
(s) improvement of optical properties of skin by light diffraction or reflection.

18. A gene panel for screening for active agents, wherein at least 50% of nucleic acids on said gene panel comprise oligonucleotides that hybridize with nucleic acids corresponding to genes selected from the group consisting of: UDP glucuronosyltransferase 2 family, polypeptide A1, complex locus; proprotein convertase subtilisin/kexin type 2; transient receptor potential cation channel, subfamily M, member 1; cholinergic receptor, muscarinic 4; deiodinase, iodothyronine, type II; glycerophosphodiester phosphodiesterase domain containing 2; histone cluster 1, H3a; oculocutaneous albinism II; tubulin polymerization promoting protein; glia maturation factor, gamma; chromosome 1 open reading frame 162; selectin L; cytochrome P450, family 7, subfamily B, polypeptide 1; CD2 molecule; interferon-induced protein 44-like; formyl peptide receptor 3; ecotropic viral integration site 2B; G protein-coupled receptor 174; Fc fragment of IgG, low affinity IIa, receptor (CD32); CD86 molecule; Serglycin; poly (ADP-ribose) polymerase family, member 14; vascular cell adhesion molecule 1; interleukin 33; major histocompatibility complex, class II, DP alpha 1; ST8 alpha-N-acetyl-neuraminide alpha-2,8-sialyltransferase 4; microRNA let-7c; KIAA0226-like; killer cell lectin-like receptor subfamily B, member 1; major histocompatibility complex, class II, DQ alpha 1; regulator of G-protein signaling 1; collagen, type V, alpha 2; sterile alpha motif domain containing 9; interferon regulatory factor 8; CD226 molecule; granzyme K (granzyme 3; tryptase II); FYN binding protein; collagen, type XIV, alpha 1; cytochrome b-245, beta polypeptide; chemokine (C-C motif) receptor 2; interferon-induced protein 44; placenta-specific 8; major histocompatibility complex, class II, DR alpha; protein tyrosine phosphatase, receptor type, C; pleckstrin; GLI pathogenesis-related 1; ribonuclease, RNase A family, k6; collagen, type III, alpha 1; cytohesin 1 interacting protein; CD53 molecule; C-type lectin domain family 7, member A; cathepsin S; fibroblast activation protein, alpha; lysozyme; secreted phosphoprotein 1; myeloid cell nuclear differentiation antigen; baculoviral IAP repeat containing 3; CD1b molecule; and collagen, type VI, alpha 6.

19. A gene panel for screening for active agents, wherein at least 75% of nucleic acids on said gene panel comprise oligonucleotides that hybridize with nucleic acids corresponding to genes selected from the group consisting of: UDP glucuronosyltransferase 2 family, polypeptide A1, complex locus; proprotein convertase subtilisin/kexin type 2; transient receptor potential cation channel, subfamily M, member 1; cholinergic receptor, muscarinic 4; deiodinase, iodothyronine, type II; glycerophosphodiester phosphodiesterase domain containing 2; histone cluster 1, H3a; oculocutaneous albinism II; tubulin polymerization promoting protein; glia maturation factor, gamma; chromosome 1 open reading frame 162; selectin L; cytochrome P450, family 7, subfamily B, polypeptide 1; CD2 molecule; interferon-induced protein 44-like; formyl peptide receptor 3; ecotropic viral integration site 2B; G protein-coupled receptor 174; Fc fragment of IgG, low affinity IIa, receptor (CD32); CD86 molecule; Serglycin; poly (ADP-ribose) polymerase family, member 14; vascular cell adhesion molecule 1; interleukin 33; major histocompatibility complex, class II, DP alpha 1; ST8 alpha-N-acetyl-neuraminide alpha-2,8-sialyltransferase 4; microRNA let-7c; KIAA0226-like; killer cell lectin-like receptor subfamily B, member 1; major histocompatibility complex, class II, DQ alpha 1; regulator of G-protein signaling 1; collagen, type V, alpha 2; sterile alpha motif domain containing 9; interferon regulatory factor 8; CD226 molecule; granzyme K (granzyme 3; tryptase II); FYN binding protein; collagen, type XIV, alpha 1; cytochrome b-245, beta polypeptide; chemokine (C-C motif) receptor 2; interferon-induced protein 44; placenta-specific 8; major histocompatibility complex, class II, DR alpha; protein tyrosine phosphatase, receptor type, C; pleckstrin; GLI pathogenesis-related 1; ribonuclease, RNase A family, k6; collagen, type III, alpha 1; cytohesin 1 interacting protein; CD53 molecule; C-type lectin domain family 7, member A; cathepsin S; fibroblast activation protein, alpha; lysozyme; secreted phosphoprotein 1; myeloid cell nuclear differentiation antigen; baculoviral IAP repeat containing 3; CD1b molecule; and collagen, type VI, alpha 6.

20. A gene panel for screening for active agents, wherein at least 90% of nucleic acids on said gene panel comprise oligonucleotides that hybridize with nucleic acids corresponding to genes selected from the group consisting of: UDP glucuronosyltransferase 2 family, polypeptide A1, complex locus; proprotein convertase subtilisin/kexin type 2; transient receptor potential cation channel, subfamily M, member 1; cholinergic receptor, muscarinic 4; deiodinase, iodothyronine, type II; glycerophosphodiester phosphodiesterase domain containing 2; histone cluster 1, H3a; oculocutaneous albinism II; tubulin polymerization promoting protein; glia maturation factor, gamma; chromosome 1 open reading frame 162; selectin L; cytochrome P450, family 7, subfamily B, polypeptide 1; CD2 molecule; interferon-induced protein 44-like; formyl peptide receptor 3; ecotropic viral integration site 2B; G protein-coupled receptor 174; Fc fragment of IgG, low affinity IIa, receptor (CD32); CD86 molecule; Serglycin; poly (ADP-ribose) polymerase family, member 14; vascular cell adhesion molecule 1; interleukin 33; major histocompatibility complex, class II, DP alpha 1; ST8 alpha-N-acetyl-neuraminide alpha-2,8-sialyltransferase 4; microRNA let-7c; KIAA0226-like; killer cell lectin-like receptor subfamily B, member 1; major histocompatibility complex, class II, DQ alpha 1; regulator of G-protein signaling 1; collagen, type V, alpha 2; sterile alpha motif domain containing 9; interferon regulatory factor 8; CD226 molecule; granzyme K (granzyme 3; tryptase II); FYN binding protein; collagen, type XIV, alpha 1; cytochrome b-245, beta polypeptide; chemokine (C-C motif) receptor 2; interferon-induced protein 44; placenta-specific 8; major histocompatibility complex, class II, DR alpha; protein tyrosine phosphatase, receptor type, C; pleckstrin; GLI pathogenesis-related 1; ribonuclease, RNase A family, k6; collagen, type III, alpha 1; cytohesin 1 interacting protein; CD53 molecule; C-type lectin domain family 7, member A; cathepsin S; fibroblast activation protein, alpha; lysozyme; secreted phosphoprotein 1; myeloid cell nuclear differentiation antigen; baculoviral IAP repeat containing 3; CD1b molecule; and collagen, type VI, alpha 6.
